(19) United States
(12) Patent Application Publication
(10) Pub. No.: US 2013/0337462 A1
(43) Pub. Date: Dec. 19, 2013

(54) EXTRACTION OF NUCLEIC ACIDS

(71) Applicant: Steffen Mergemeier, Berlin (DE)

(72) Inventor: Steffen Mergemeier, Berlin (DE)

(21) Appl. No.: 13/995,859

(22) PCT Filed: Dec. 20, 2011
(86) PCT No.: PCT/EP11/73352

§ 371 Date: Sep. 3, 2013

(30) Foreign Application Priority Data
Dec. 20, 2010 (EP) 10195963.3

(51) Int. Cl. C12Q 1/68

(52) U.S. Cl. CPC C12Q 1/6806
USPC 435/6.12

(57) ABSTRACT

The invention relates to a nucleic acid extraction material which comprises at least one ion exchange material and PVPP.

The invention relates to a method for extraction of nucleic acids by means of a nucleic acid extraction material, which comprises at least one ion exchange material and PVPP. Furthermore, the invention relates to the use of the extraction material and to a kit for nucleic acid extraction.

A polymerase chain reaction (PCR Polymerase chain reaction) is one of the most important biochemistry methods for amplification of nucleic acids. With this technique, nucleic acids can be amplified very quickly and effectively, and subsequently sequenced or detected. An important field of use of PCR is clinical diagnostics. Here, the technique is used, among other things, for the detection of pathogens such as noroviruses. Another field of use is food analysis. Thus it is possible, for example, to detect contamination with salmonellae or the presence of allergens in food samples.

Another sector of PCR is analysis with regard to pathogenic microorganisms in foods. Thus, EP 0 643 140 B1, for example, describes a method for the detection of nucleic acids by means of the detection of the amplification product of the PCR, using a pigment compound. The pigment does not fluoresce in the free state. Fluorescence can only be determined during the reaction with a double-strand nucleic acid. In this way, it is possible to determine the type and number of bacteria in a sample.

Pathogenic microorganisms that are detected in foods by means of PCR methods are, for example, Listeria monocytogenes, Campylobacter jejuni, and salmonellae. Food contaminated with pathogenic microorganisms causes illnesses such as salmonellosis or listeriosis. Regular inspections of product batches are necessary so that foods that carry germs are not brought into commerce. Conventional microbial Salmonella detection in foods required more than three days due to long accumulation times. Real-time PCR as a sensitive detection method, in contrast, offers the advantage that a result is already determined after one day. Analysis of foods such as milk, dairy products and eggs, as well as egg products and meat, is absolutely necessary. Furthermore, it is important to detect not only bacteria but also specific viruses in foods. Noroviruses are considered to be the cause of acute gastroenteritides. The noroviruses belong to the family of the Calciviridae and are single-strand RNA viruses without an envelope, which occur worldwide. The characteristic illness profile of acute gastroenteritis includes severe nausea, followed by sudden vomiting and diarrhea, rarely with fever. After 12 to 60 hours, the virus has been eliminated from the body. Treatment takes place purely symptomatically and consists of providing sufficient fluids and electrolytes. The noroviruses are excreted via the stool and vomit. In this connection, the stool contains a particularly high virus concentration; for an infection, a dose of fewer than 100 virus particles is sufficient. The virus is highly infectious and stable in the environment. Contagion takes place by the fecal-oral route, for example by means of hand contact with contaminated surfaces or by oral intake of droplets that contain the virus in cases of severe vomiting. Detection of noroviruses can take place, on the one hand, with ELISA or by way of the detection of nucleic acids. Detection using the PCR technique makes reverse transcriptase necessary. The transcripted RNA is multiplied and detected as cDNA (complementary deoxyribonucleic acid). Detection of noroviruses by means of RT-PCT is clearly more sensitive and more specific than detection with an ELISA.

For the examination of food samples or other samples using PCR, precise sample preparation and nucleic acid purification as well as removal of inhibitors are very important, because inhibitory substances impair the success of the PCR, in that they suppress amplification, inactivate the Taq polymerase, or suppress the measurement signal.

Purification of nucleic acids on solid phases, which are based on a silica matrix, is a technique used in many commercial kits. The principle of purification is based on binding of the nucleic acid to the solid phase, as a function of the pH and of the salt concentration of the buffer. Under chaotropic conditions, the network of the hydrogen bridge relationships in water is destroyed. As a result, the formation of a hydrate envelope around the macromolecules (DNA, RNA) is cancelled out. In the absence of chaotropic ions, a hydrate envelope forms once again, so that the interaction between silica membrane and macromolecule is cancelled out. Technically, this method of purification was implemented in the spin-filter method, on the one hand, and in the magnetic beads technology, on the other hand.

Nucleic acid extraction is disclosed in WO 1996/09404, for example. Fundamentally, the extraction takes place in four steps: cell lysis, bonding of the nucleic acids to a matrix, as well as washing and elution of the nucleic acids. A disadvantage in this connection is that the extraction is very time-consuming, since numerous washing steps have to be carried out, for example, in which nucleic acid is constantly washed out. As a result, the yield of nucleic acid is significantly reduced. Furthermore, this extraction method is difficult to automate.

A further variant for the isolation of nucleic acids with a silica matrix is disclosed in WO 1998/031840, for example, in which magnetic silica particles are used. In this connection, molecules with a large surface area are used, which possess a magnetic moment when they are exposed to a magnetic field. Among other things, porous glass types surface-modified with colloidal magnetite (Fe₃O₄) are used. These magnetic beads and a special binding buffer are added to the sample after lysis. The nucleic acids bind to the silica matrix. By applying a magnetic field, the beads collect at the edge of the vessel, and the contaminants can be removed in multiple washing steps. The target molecules detach by removal of the magnet and addition of the elution buffer. If a new magnetic field is applied, the elution buffer with nucleic acid can be separated from the beads. The advantage of this technique consists in the high degree of automation capacity of the work sequences, with little apparatus effort.

In the state of the art, methods are furthermore described with which bacteria can be detected in samples. Thus, for example, EP 1 574 584 B1 describes a method for the isolation of bacteria from biological samples. The methods are suitable for sample preparation of biological samples for methods based on nucleic acid or immune-diagnostic methods for the detection of bacteria. In this connection, bacteria are detected and isolated by means of a specific antibody.

It is a disadvantage of the state of the art that the amount of the nucleic acid to be isolated is also reduced by the numerous purification steps, and therefore the yield is significantly reduced. The numerous purification steps furthermore lead to the result that the extraction takes a very long time. Furthermore, the methods described in the state of the art cannot be automated, or can be automated only with difficulty. Furthermore, extraction cannot be carried out with all samples. Highly complex samples that contain numerous components, such as stool samples, for example, are difficult to process, because the nucleic acid is frequently damaged during the purification process.

Therefore it was the task of the invention to make available a nucleic acid extraction that does not demonstrate the disadvantages or defects of the state of the art.

This task is accomplished by means of the independent claims. Advantageous embodiments are evident from the dependent claims.

It was completely surprising that a nucleic acid extraction material can be made available, which does not have the disadvantages of the state of the art and comprises at least polyvinylpolypyrrolidone (PVPP) and an ion exchange material. The extraction material according to the invention allows rapid and material-saving extraction or purification of nucleic acids. The extraction material can also be advantageously utilized for automation processes. Furthermore, it can be used universally and does not have to be adapted to individual applications.

The ion exchange material is preferably selected from the group comprising Sephadex, Chelex, zeolites and/or Sepharose. Sephadex, in the sense of the invention, particularly describes a three-dimensionally cross-linked polysaccharide, which is obtained by means of transverse cross-linking of the linear macromolecules of dextran. Sephadex is indifferent to cations and anions and contains many hydroxy groups, making it strongly hydrophilic and causing it to swell in water or an electrolyte solution. In a preferred embodiment, Sephadex can be conjugated with functional groups, comprising diethylaminoethyl, diethyl-2-hydroxypropylaminoethyl, carboxymethyl or sulfopropyl groups. Sephadex is advantageously selected from the group comprising Sephadex G-10, Sephadex G-100, Sephadex G-15, Sephadex G-25, Sephadex G-50 and/or Sephadex G-75. Chelex, in the sense of the invention, also refers to a polymer that preferably binds ions. Zeolites, in the sense of the invention, are crystalline alumosilicates. The crystal lattices of the zeolites are particularly composed of SiO₄ and AlO₄ tetrahedrons, which are linked with one another by way of oxygen bridges and preferably form rings or prisms. These in turn connect to form further secondary building units (secondary building units, SBU), which can contain up to 16 Si or Al atoms, in each instance, leading to great structural variety. In this connection, a spatial arrangement of cavities having the same construction is formed, which cavities are accessible by way of windows (pore openings) or three-dimensional channel systems. It has been shown that different elements, comprising ions, proteins, hydrocarbons or fats interact with zeolites, and can be separated by nucleic acids. It can furthermore be advantageous to use Sepharose as an ion exchange material. Sepharose, in the sense of the invention, refers to a modified polysaccharide on an agarose basis, the polysaccharide chains of which are linked to form a three-dimensional network.

PVPP, in the sense of the invention, refers, in particular, to polyvinylpolypyrrolidones (including crospovidone, derived from cross-linked polyvinylpyrrolidone), which is particularly formed when heating vinylpyrrolidone with alkalis or divinyl compounds. PVPP is a cross-linked polymer that is essentially insoluble in water and all solvents. PVPP is advantageously a copolymer that consists of vinyl monomers. It can be preferred to use another copolymer that consists of vinyl monomers instead of PVPP or in combination with PVPP as a preferred nucleic acid extraction material. A copolymer that consists of vinyl monomers preferably has the general formula

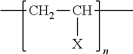

In this connection, X stands for a hetero atom or a group fixed in place by way of a hetero atom. Preferred groups are shown in the following table:

TABLE 1

| X | Name |
|---|---|
| —OH | Polyvinyl alcohol (from polyninyl acetate) |
| —Halogen | Polyvinyl chloride, polyvinyl fluoride, etc. |
| —O—CO—R | Polyvinyl ester |
| 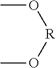 | Polyvinyl acetate |
| —O—R | Polyvinyl ether |
| —P(O)(OH)₂ | Polyvinyl phosphonic acids |
| —C₆H₅ | Polystyrene |
| —SO₃H | Polyvinyl sulfonic acids |
| —NH₂ | Polyvinyl amines |
| 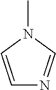 | Polyimidazoles |
| 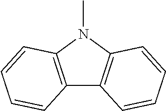 | Polyvinylcarbazoles |
| 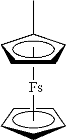 | Polyvinylferrocenes |

The preferred homopolymers or copolymers particularly comprise monomers of the structure H₂C═CH—X, which consist of a polymerizable vinyl group and a substituent X, which in turn can consist of only a single atom (e.g. F (vinyl fluoride), Cl (vinyl chloride), Br (vinyl bromide), or an atom group. Examples of the latter are X=alkyl (1-alkenes), aryl (for example styrene), OR (vinyl ether), O—CO—R (vinyl ester), COOR (acrylic acid and its esters), CONR₂ (acrylamides), CN (acryl nitrile), NR₂ (vinyl amine), NH—CO—R (vinyl amide), SO₃H (vinyl sulfonic acid), PO(OH)₂ (vinyl phosphonic acid), and others. Monomers with the structural unit C═C—C═O, in other words 1,3-dienes, are also essentially vinyl monomers. However, they are also referred to as diene monomers. Furthermore, divinyl and polyvinyl monomers such as divinyl benzene, for example, also belong to the vinyl monomers.

In a broader sense, monomers with C, C double bonds, for example of the type H₂C═CR¹R², for example vinylidene chloride (R¹═R²═Cl), are also considered to be vinyl monomers in the sense of the invention. Finally, compounds R¹R²C═CR³R⁴ with only one or finally no hydrogen atom directly bound to the unsaturated carbon atoms are also substituted vinyl monomers.

The extraction material can advantageously be present as a loose powder, tablet, pellet or chromatography column fill material, which comprises at least one ion exchange material and a PVPP component. It is preferred that the loose powder, the tablet, the pellet or the chromatography column fill material contains the ion exchange material and the PVPP component in a grain size fraction of 5-1000 μm, preferably in a range of 50-250 μm.

It was completely surprising that it was possible to bind components from samples, by means of the extraction material according to the invention, with the nucleic acid to be isolated not binding. It is advantageous if the extraction material is used for sample treatment, particularly for treatment of samples, particularly nucleic acid samples for PCR analysis.

A sample is preferably a culture medium, a bodily fluid and/or a mixture of material of plant and/or animal origin. Surprisingly, it was also possible to extract nucleic acids from food samples. A person skilled in the art knows that bodily fluids comprise, for example, stool, saliva, blood, lymph, urine, synovial fluid, digestive juices, secretions, excretions or liquid excretions or other fluids of an organism. The sample can be present in liquid or solid form, whereby a solid form, in the sense of the invention, particularly also comprises frozen samples or tissue samples or bone samples. A person skilled in the art knows that microorganisms are preferably cultured in a culture medium, with the culture medium containing all the nutrients relevant for microorganisms. The culture medium can be present in solid or liquid form, and particularly serves for culturing microorganisms comprising bacteria, Archaea, fungi, microalgae, protozoa, and viruses.

The nucleic acid or the nuclein sample preferably comprises RNA and/or DNA. RNA refers to the elongated molecule consisting of nucleotides, which, in the cell, mainly has the function of implementing the genetic information stored in the deoxyribonucleic acid (DNA). Various forms of RNA participate in this: mRNA, which provides the information for protein biosynthesis (translation) as a copy of the gene, rRNA, which is represented in the ribosomes in the form of different species (5S, 16S, 23S in bacteria and 5S, 8S, 18S, 28S in higher organisms), and tRNA, which mediates the installation of the activated amino acids into the growing protein chain on the ribosomes. In the cell nucleus, there is also heterogeneous nuclear RNA (hnRNA from heterogeneous nuclear RNA), which consists of precursors of mRNA, as well as small nucleus RNA (snRNA), which takes part in joining the exons of the RNA. RNA molecules can also possess enzymatic activities (ribozymes) or take on regulatory functions by means of RNA interference (siRNA, miRNA). Such RNAs are frequently grouped under the term ncRNA. In the case of RNA viruses, the RNA itself is the carrier of the genetic information. It was completely surprising that RNA comprising mRNA, tRNA, rRNA, snRNA, miRNA, virus RNA, or hnRNA can be extracted from a sample by means of a preferred extraction material. Advantageously, DNA can also be isolated from a sample.

DNA, in the sense of the invention, refers, in particular, to long-chain polynucleotides that contain the main genetic information (the genome) of living beings, stored in them. The main amount of the DNA is contained in eukaryotes in the cell nucleus, specifically in the chromosomes or in the chromatin. In the case of bacteria, it is not found in a separate cell organelle and mainly consists of a single molecule closed in ring shape. Bacteria contain not only the genomic DNA but also smaller, also ring-shaped DNA molecules, the easily transferred plasmids. It was completely surprising that DNA can be isolated easily and quickly by means of the nucleic acid extraction material. The DNA is essentially not damaged and can therefore be passed to subsequent analysis as a whole. This represents a significant advantage as compared with the state of the art, because damage to the DNA permits only incomplete analysis. More comprehensive analyses can be performed by means of the extraction material.

The invention therefore also comprises a method for purification of a nucleic acid present in a sample, on an analytical or preparative scale, comprising a matrix comprising
a. a synthetic or natural ion exchange material, and
b. a cross-linked polymer with a pyrrolidone structure,
   wherein the sample is brought into contact with the matrix and particularly undesirable components of the sample, comprising proteins, salts, hydrocarbons, ions and/or fats interact with the matrix. In this way, rapid extraction of the nucleic acid is possible, with the separation, purification and/or detection of the nucleic acid preferably being carried out in one method step, and, advantageously, no washing and/or elution steps being required. The method for sample preparation for the extraction of nucleic acids from a sample comprises the following steps:
bringing the sample into contact with a lysis buffer,
heating the mixture of sample and lysis buffer,
centrifuging the mixture,
taking up the top fraction, and
bringing the top fraction into contact with the nucleic acid extraction material.

By means of the method, nucleic acids can advantageously be extracted from samples, preferably bodily fluids, plant or animal samples and/or culture media. Thus, it is surprisingly possible to extract bacterial or viral nucleic acids from stool samples, allergy-associated nucleic acids from plant or animal samples and/or nucleic acids from culture media. No binding of the nucleic acids to a membrane or other material is necessary, and therefore no washing or elution steps have to be carried out, and therefore the yield of the isolated nucleic acid can be significantly improved. Furthermore, the method can be carried out easily on a small or large scale, also completely or partially in automated form.

The sample to be studied is brought into contact with a lysis buffer in a first step. Lysis, in the sense of the invention, refers particularly to dissolution (lysis) of cells, with the action of lytic enzymes (lysozymes) and destruction of the cell membrane (cytolysis). However, it can also be preferred that the sample is solubilized by means of mechanical methods, such as ultrasound treatment, French Press, or glass-bead mill. However, a lysis buffer is preferred, because nucleic acids are damaged less in this way. In a preferred embodiment, the lysis buffer comprises a buffer that adjusts a preferred pH range.

It is preferred that the lysis buffer comprises Triton, preferably Triton X-15, X-35, X-45, X-100, X-102, X-104, X-114, X-165, X-305, X-405 and/or X-705. Triton is a non-ionic surfactant from the group of octylphenol ethoxylates, which denatures proteins. Surfactants are amphiphilic (bifunctional) compounds having at least one hydrophobic and one hydrophilic molecule. The hydrophobic radical is, in most cases, a hydrocarbon chain that is as linear as possible and preferably has 8 to 22 carbon atoms. The hydrophilic radical is a polar head group that carries either a negative or positive electrical charge (can be hydrated) or is neutral. Surfactant betaines or amino acid surfactants (amphoteric or hybrid-ionic surfactants) carry negatively and positively charged groups in one molecule. Advantageous properties of surfactants are oriented adsorption on border surfaces and aggregation to form micelles and the formation of lyotropic phases. Non-ionic surfactants demonstrate a non-charged head group that brings about solubility in water.

It was completely surprising that the nucleic acid extraction yield is significantly improved by means of the buffer, to which Triton was added. Furthermore, the purified nucleic acid demonstrates great purity, i.e. it is essentially not contaminated with any further components. The purity of the nucleic acid plays an important role, particularly in further analysis methods, because numerous enzymes, such as DNA polymerase, for example, are impaired by contaminants, in terms of their function. In this way, false results can come about. A person skilled in the art knows that the number and availability of biological samples are often limited, and that each sample must be used carefully. Accordingly, the extraction material according to the invention can be viewed as technical progress, because the nucleic acid is not damaged during extraction and therefore represents an optimal starting material for subsequent analyses. Furthermore, no additional precipitation reactions are required to remove further contaminants—the extracted nucleic acid can be used further in this form.

After addition of the lysis buffer, it can be preferred to warm or heat the mixture of the lysis buffer and the sample, in order to ensure complete lysis of the cellular components. It has proven to be extremely advantageous to warm/heat the mixture, preferably stool, to 80° C. to 95° C., preferably 90° C., for 5 minutes to 15 minutes, particularly preferably for 7 to 10 minutes.

At the preferred temperatures, essentially complete denaturing of the proteinogenic components of the sample is achieved, with the nucleic acid not being damaged.

After heating, the sample is preferably centrifuged, with a Triton phase that comprises not only non-dissolved sample components but also non-polar components forming at room temperature. Furthermore, an aqueous phase—a top fraction—advantageously forms, in which nucleic acid is present. This top fraction can be applied to a centrifuge filter and centrifuged, for example. A preferred filter is a spin filter, for example. In a preferred embodiment of the invention, the filter comprises the nucleic acid extraction material, particularly PVPP and an ion exchange material. The nucleic acid penetrates the filter and is present in solution, with all the inhibiting components being bound by the ion exchange material and PVPP, so that they cannot penetrate the filter. It can be preferred to concentrate the nucleic acid after separation, purification and/or detection. Concentration can take place by means of vacuum evaporation or precipitation, for example.

In a preferred embodiment, the extraction material is present as a tablet, pellet or chromatography column fill material, which comprises at least one ion exchange material and a PVPP component. It was completely surprising that the extraction material can be structured as a tablet. In this way, easy transport and storage of the extraction material is possible. Advantageously, bringing into contact takes place in such a manner that the top fraction is brought into contact with a tablet comprising PVPP and ion exchange material, and that the mixture obtained in this manner is centrifuged once again and the top fraction is used, in particular, for a PCR reaction. After lysis of the sample, the tablet can be added to the top fraction. In this way, rapid extraction of the nucleic acid is possible. It can furthermore be preferred that the lysis buffer and/or ion exchange material is/are present as a tablet. The lysis buffer in tablet form is added to the sample; the tablet dissolves and lyses the sample. The ion exchange material tablet can advantageously be introduced into the top fraction with the PVPP.

It can also be preferred, however, that bringing the top fraction into contact with the nucleic acid extraction material takes place in such a manner that the top fraction is applied to a chromatography column comprising the nucleic acid extraction material, and that the eluate obtained by means of the column is used in a PCR method. Chromatography, in the sense of the invention, refers, in particular, to the physical/chemical separation of substance mixtures on the basis of the different distribution between a stationary and a mobile phase. The individual components are delayed differently as the result of differently strong interactions with the stationary phase in comparison with the mobile phase; they therefore travel different distances during the same time. Preferred chromatography methods are, for example: thin-layer chromatography, gas chromatography, high-performance or high-pressure liquid chromatography (HPLC), ion chromatography, and column liquid chromatography. Rapid and efficient purification of the sample can be achieved by means of chromatography, because the nucleic acid extraction material, comprising ion exchange material and PVPP, is present in the column as chromatography column fill material. As a result, the purity of the extracted nucleic acid is very high after chromatography. Essentially, all the inhibitory or disruptive components can be removed from the sample, by means of chromatography, from the nucleic acid. The components are preferably retained in the column, with the nucleic acid passing through the column.

If only a specific nucleic acid is to be analyzed, digestion can take place by means of adding enzymes. If only RNA is to be analyzed, the DNA can be digested by means of DNases and removed. Vice versa, the RNA can be digested by means of RNases, if only the DNA is to be analyzed.

In a preferred embodiment, the extraction material is used for sample treatment, particularly for treatment of nucleic acid samples for PCR analysis. A person skilled in the art knows that preparation of nucleic acid samples corresponds to extraction of nucleic acids from a sample. PCR preferably comprises long range PCR, nested PCR, inverse PCR, anchored PCR, RT-PCR, and quantitative RT-PCR (also called real-time PCR).

Particularly in the case of PCR analysis, it is important that inhibiting components be removed from the samples, since the method of functioning of PCR is impaired otherwise. PCR inhibitors are fundamentally grouped in three categories, depending on their method of effect: (1) inactivation of DNA polymerase, (2) decomposition of the nucleic acids, and (3) a negative influence on lysis. Substances that have an inhibitory effect comprise salts, ions (e.g. calcium ions), fats or proteins. However, compounds that are used for sample preparation, such as ethanol or detergents such as sodium dodecylsulfate, for example, also demonstrate inhibitory effects. It was completely surprising that inhibitory components can be easily and quickly separated from the nucleic acid. It is advantageous that the nucleic acid does not have to be eluted from a matrix by means of numerous elution steps, but rather the components are bound and the nucleic acid is preferably present in solution.

Separation or binding of the components that could inhibit a subsequent PCR particularly takes place by means of two mechanisms. The first mechanism comprises binding of non-polar compounds in an aqueous phase of the sample, particularly by means of PVPP, which forms a non-aqueous phase under defined conditions. By means of the second mechanism, inhibitors are removed from the aqueous phase by means of separation using differences in molecular weight, by means of the ion exchange material.

It is advantageous that a nucleic acid extracted by means of the extraction material can also be characterized by other analysis or quantification methods. For example, it can be preferred to subject the extracted nucleic acid to restriction digestion, in which the nucleic acid is cut by restriction enzymes at defined positions. The cut nucleic acid can subsequently be analyzed by means of gel electrophoresis, for example. Analysis of the extracted nucleic acid by means of microarray methods or other high-throughput screening methods is preferred.

The invention furthermore relates to a kit for nucleic acid extraction, which comprises at least one ion exchange material and PVPP. With the kit, nucleic acids can be easily and quickly extracted from a sample. Advantageously, the kit can also comprise a lysis buffer, particularly in tablet form. The ion exchange material can also be present in tablet form.

The invention leads to numerous advantages, including:
more nucleic acid can be extracted, because it does not bind to a matrix, and nucleic acid is not lost as the result of incomplete binding;
no discrimination between different components of the sample;
simple and quick implementation, with a reduced number of work steps;
low costs for the extraction material, because no washing solutions or elution solutions are required; and
the extraction material has a simple structure—only PVPP and an ion exchange material are required.

The invention can also be viewed as a combination invention. The combination of the known elements of ion exchange material and PVPP leads to surprising effects in the extraction of nucleic acids. Only the combination of these compounds leads to synergistic advantages that bring about efficient separation of the inhibitory components of the nucleic acids. The components bind to the combination of ion exchange material and PVPP, where the nucleic acids do not interact with the ion exchange material or PVPP, and therefore can be easily separated from the components.

1. Nucleic acid extraction material, comprising at least polyvinylpolypyrrolidone (PVPP) and an ion exchange material.

2. The extraction material according to claim 1,
wherein
the ion exchange material is selected from the group consisting of Sephadex, Chelex, zeolites, Sepharose and combinations thereof.

3. The extraction material according to claim 1,
wherein
extraction material is present as a loose powder, tablet, pellet or chromatography column fill material, which comprises at least one ion exchange material and a PVPP component.

4. The extraction material according to claim 3,
wherein
the loose powder, the tablet, the pellet or the chromatography column fill material contains the ion exchange material and the PVPP component in a grain size fraction of 5-1000 μm, preferably in a range of 50-250 μm.

5. Method for sample preparation comprising providing of the extraction material according to claim 1 and preparing a sample with said extraction material, particularly for preparation of samples for PCR analysis.

6. The method of claim 5, wherein the sample is a culture medium, a bodily fluid and/or a mixture of material of plant and/or animal origin.

7. A method for purification of a nucleic acid present in a sample, on an analytical or preparative scale, comprising a matrix comprising
a. a synthetic or natural ion exchange material, and
b. a cross-linked polymer with a pyrrolidone structure,
wherein the sample is brought into contact with the matrix and undesirable components of the sample, comprising proteins, salts, hydrocarbons, ions and/or fats interact with the matrix.

8. The method according to claim 7, wherein the purification of the nucleic acid is carried out in one method step.

9. Method for the extraction of nucleic acids from a sample according claim 7,
wherein
the method comprises the following:
bringing the sample into contact with a lysis buffer,
heating the mixture of sample and lysis buffer,
centrifuging the mixture,
taking up the top fraction, and
bringing the top fraction into contact with the nucleic acid extraction material according to claim 1 to 4.

10. The method according to claim 9,
wherein
bringing the top fraction into contact with the nucleic acid extraction material takes place in such a manner that the top fraction is applied to a chromatography column comprising the nucleic acid extraction material, and that an eluate obtained via the column is used in a PCR method.

11. The method according to claim 7,
wherein
bringing the to fraction into contact takes place in such a manner that the top fraction is brought into contact with a tablet comprising PVPP and ion exchange material, and that the mixture obtained in this manner is centrifuged once again and the top fraction is used for a PCR reaction.

12. The method according to claim 7,
wherein
the lysis buffer comprises Triton.

13. The method according to claim 7,
wherein
warming/heating of the mixture of the lysis buffer and the sample takes place at 80° C. to 95° C. for 5 minutes to 15 minutes.

14. The method according to claim 7,
wherein
the sample comprises bodily fluids, plant or animal samples and/or culture media.

15. The method according to claim 7,
wherein
the nucleic acid is concentrated after separation, purification and/or detection.

16. Kit for nucleic acid extraction, comprising, in one container, at least one ion exchange material and PVPP according to claim 1 and, in another container instructions how to use the kit.

17. The extraction material according to claim 4,
wherein the PVPP component is in a grain size fraction in a range of 50-250 μm.

18. The method according to claim 12,
wherein
the lysis buffer comprises Triton X-15, X-35, X-45, X-100, X-102, X-104, X-114, X-165, X-305, X-405 and/or X-705.

19. The method according to claim 13,
wherein the sample is stool.

20. The method according to claim 13,
wherein warming/heating of the mixture of the lysis buffer and the sample takes place at 90° C. and/or for 7 to 10 minutes.
